(19) United States
(12) Patent Application Publication
(10) Pub. No.: US 2025/0017827 A1
(43) Pub. Date: Jan. 16, 2025

(54) Acid-Stable Glass Fillers And Self-Adhesive Dental Composite Cements With Good Transparency Containing Them

(71) Applicant: Ivoclar Vivadent AG, Schaan (LI)

(72) Inventors: Christian RITZBERGER, Grabs (CH); Norbert MOSZNER, Triesen (LI); Yohann CATEL, Sevelen (CH); Marc DITTMER, Feldkirch (AT); Alexandros GIANASMIDIS, Balgach (CH)

(21) Appl. No.: 18/761,692

(22) Filed: Jul. 2, 2024

(30) Foreign Application Priority Data
Jul. 4, 2023 (EP) 23183404.5

(51) Int. Cl. A61K 6/889; A61C 5/73; A61K 6/20; A61K 6/62; A61K 6/77

(52) U.S. Cl. CPC A61K 6/889; A61C 5/73; A61K 6/20; A61K 6/62; A61K 6/77; A61C 2201/005

(57) ABSTRACT

Acid-stable glass fillers and self-adhesive dental composite cements with good transparency containing them. X-ray opaque glass filler with the following composition (wt. %): SiO₂: 54.0-64.0; Al₂O₃: 10.0-25.0; B₂O₃: 0.0-10.0; La₂O₃: 0.1-10.0; WO₃: 5.0-14.0; ZrO₂: 0.1-7.0; MgO: 1.0-7.9; CaO: 0.1-6.0; ZnO: 0-6.0; SrO: 0-6.0, and radically polymerizable composition comprising, in addition to the radiopaque glass filler, at least one radically polymerizable monomer without an acid group, at least one radically polymerizable monomer containing an acid group, and at least one initiator for the radical polymerization. The glass filler is characterized by a high acid stability.

### CROSS-REFERENCE TO RELATED APPLICATIONS

This application claims priority to EP 23183404.5, filed on Jul. 4, 2023, the disclosure of which is incorporated herein by reference in its entirety.

### TECHNICAL FIELD

The invention relates to acid-stable, radiopaque glass fillers and radically polymerizable, self-adhesive composites with good transparency, which are particularly suitable as dental materials, e.g. as dental cements, filling composites or veneering materials and for the production of inlays, onlays or crowns.

### BACKGROUND

Composites are mainly used in the dental field for the fabrication of direct and indirect fillings, i.e. as direct and indirect filling composites, as well as cements. The polymerizable organic matrix of the composites usually consists of a mixture of monomers, initiator components and stabilizers. Mixtures of dimethacrylates are usually used as monomers, which may also contain monofunctional and functionalized monomers. Frequently used dimethacrylates are 2,2-bis[4-(2-hydroxy-3-ethacryloyloxypropyl)phenyl]propane (Bis-GMA) and 1,6-bis[2-methacryloyloxyethoxycarbonylamino]-2,2,4-trimethylhexane (UDMA), which have a high viscosity and yield polymers with good mechanical properties and low polymerization shrinkage. The main reactive diluents used are triethylene glycol dimethacrylate (TEGDMA), 1,10-decanediol dimethacrylate (D₃MA) or bis(3-methacryloyloxymethyl)tricyclo-[5.2.1.0^{2,6}] decane (DCP). Monofunctional methacrylates, such as p-cumylphenoxyethylene glycol methacrylate (CMP-1E), are also suitable for reducing viscosity and further cause a reduction in network density and increased double bond conversion.

To produce self-adhesive composites, strongly acidic adhesive monomers are used, such as 10-methacryloyloxydecyl dihydrogen phosphate (MDP), which etches the tooth structure and causes adhesion to enamel/dentin through ionic relationship. Adhesive monomers impart self-adhesive properties to composites and thus allow the composites to be used without pre-treatment of the tooth structure with an enamel/dentin adhesive, which makes their use particularly attractive.

In addition to the organic matrix, composites contain one or more fillers, which are usually surface-modified with a polymerizable adhesion promoter, such as 3-meth-acryloyloxypropyl trimethoxy silane. Fillers improve the mechanical properties (strength, modulus of elasticity, abrasion resistance) and the processing properties (paste consistency, sculptability) of the materials and impart x-ray opacity.

It is problematic that acidic adhesive monomers often interact adversely with fillers. For example, acidic adhesive monomers are bound to the filler surface by the formation of insoluble salts, or they form poorly soluble salts during storage with ions dissolved from the fillers. This leads to a significant reduction of the adhesive monomer concentration in the resin matrix, which is associated with a reduction or even a loss of the adhesive properties. Composites with acidic adhesive monomers therefore have only limited storage stability.

The curing of methacrylate based dental materials is achieved by radical polymerization, using photoinitiators, thermal initiators or redox initiator systems, depending on the field of application. Dual-curing systems contain a combination of photoinitiators and redox initiators.

Composite cements usually contain redox systems because they ensure sufficient curing even when light curing is not possible due to insufficient transmittance. Mostly, redox initiator systems are used that are based on a mixture of dibenzoyl peroxide (DBPO) with tertiary aromatic amines, such as N,N-diethanol-p-toluidine (DEPT), N,N-dimethyl-sym.-xylidine (DMSX) or N,N-diethyl-3,5-di-tert.-butylaniline (DABA). Since the radical formation of DBPO/amine-based redox initiator systems is very much impaired by strong acids and thus also by strongly acidic adhesive monomers, redox initiator systems containing cumene hydroperoxide are preferably used in combination with thioureas, such as acetyl thiourea.

In order to ensure sufficient storage stability of the redox initiators, redox initiator system-based materials are usually used as so-called 2-component systems (2K), whereby the oxidizing agent (peroxide or hydroperoxide) and the reducing agent (amines, sulfinic acids, barbiturates, thioureas, etc.) are incorporated into spatially separated components. These are mixed together only shortly before use. Double-push syringes, which have separate cylindrical chambers to hold the components, are increasingly being used for mixing. The components are pushed out of the chambers simultaneously with two connected pistons and mixed together in a nozzle. In order to obtain mixtures that are as homogeneous as possible, it is advantageous to mix the components together in approximately equal volume proportions.

Conventional luting cements, such as ZnO eugenol cements, zinc phosphate cements, glass ionomer cements (GIC) and resin modified glass ionomer cements (RMGI) are not suitable for use with double-push syringes because they contain a powder component, which makes mixing the components much more difficult. In addition, glass ionomer cements have only low transparency and relatively poor mechanical properties.

Conventional glass ionomer cements (GIC) contain an aqueous solution of a high molecular weight polyacrylic acid (PAA, number average molar mass greater than 30,000 g/mol) or a copolymer of comparable molar mass of acrylic acid and itaconic acid as the liquid component and a calcium-fluorine-aluminum glass as the powder component. After mixing the components, they cure by ionic ionomer formation only. The disadvantages of glass ionomer cements are their low transparency and poor mechanical properties. RMGI additionally contain hydrophilic monomers, such as 2-hydroxyethyl methacrylate (HEMA). They cure both by an acid-base reaction and by radical polymerization. They are characterized by improved flexural strength compared to conventional GIC.

U.S. Pat. No. 8,053,490 B2, which is hereby incorporated by reference, discloses fluoride-releasing dental materials containing a fluoroaluminosilicate glass filler (FAS) reacted with an aqueous solution of an acid group-containing monomer or oligomer. The acid group-containing monomer or oligomer is thereby bound to the filler surface and is intended to prevent a reaction of the filler with reactive components of the cement. The materials are characterized by low transparency and unsatisfactory mechanical properties.

JP 2016030741 A1 discloses dental materials containing as a filler a fluoro-boro-aluminosilicate glass surface-modified with a silane and a polymeric carboxylic acid. The fillers have a spherical structure and are said to exhibit sustained release of fluoride ions and other ions. The dental materials show no self-adhesion to dentin and enamel and have only unsatisfactory mechanical properties.

US 2016/0324729 A1, which is hereby incorporated by reference, discloses fillers for glass ionomer cements whose surface is first silanized and then modified with an unsaturated carboxylic acid, which is bonded to the filler surface via a silicon atom. The carboxyl group of the carboxylic acid is supposed to interact with the constituents of the glass ionomer cement and stabilize the cement in this way. The materials have only moderate transparency and poor mechanical properties.

CN 102976618 A discloses low-cost glass fillers for water-based glass ionomer cements containing 25-35 wt. % Al₂O₃, 30-45 wt. % SiO₂, 2-8 wt. % Na₂O, 5-15 wt. % CaF₂ and 5-8 wt. % SrO and/or CaO. The glass powders are treated with hydrochloric acid or acetic acid at room temperature and then dried. They are said have a stable quality and good mechanical properties and continuously release fluoride ions. The glass ionomer cements have only low transparency and poor mechanical properties.

Dai et al, Int. J. Nanomedicine 14 (2019) 9185, have shown that surface treatment of basic ZrO₂ fillers with 10-methacryloyloxydecyl dihydrogen phosphate (MDP) can improve the flexural strength of dental composites and that prior coating of ZrO₂ particles with Zr(OH)₄ improves the bonding of MDP to zirconia. Dental materials based on the surface-treated fillers are not self-adhesive and have only a low transparency.

According to U.S. Pat. No. 8,071,662 B2, which is hereby incorporated by reference, surface modification of basic fillers, such as ZrO₂, with a strong acid, such as 3-methacryloyloxypropyl sulfonic acid, is said to improve the storage stability of self-etching dental materials. The disclosed dental materials are not self-etching and have unsatisfactory transparency.

DE 24 46 546 A1 discloses the treatment of naturally occurring silicon dioxide with a hydrous mineral acid to remove acid-soluble impurities. The silicon dioxide is said to be suitable as a filler for dental purposes. Radiopaque or self-adhesive dental materials are not disclosed.

US 2001/0034309 A1, which is hereby incorporated by reference, discloses the treatment of inorganic fillers with peracids or peracid salts to remove organic substances, e.g. carbon. The removal of organic substances is said to improve the bonding of silane adhesion-promoting agents to the filler surface and thus the integration of the fillers into the organic matrix of radically polymerizable dental materials. Self-adhesive materials are not disclosed.

### SUMMARY

It is an object of the present invention to provide acid-stable, radiopaque glass fillers and storage-stable, self-adhesive dental composites with good transparency and good mechanical properties, which can be mixed and applied well as 2-component systems using double-push syringes. The composites should be particularly suitable for use as dental luting cements, be radiopaque and easy to prepare.

### BRIEF DESCRIPTION OF THE DRAWING

FIG. 1 shows the decrease in the concentration of the acid monomer MDP as a function of the storage time in composite pastes with an acid stable radiopaque glass filler.

### DETAILED DESCRIPTION

This object is achieved by glass filler with the following composition (wt. %): SiO₂: 54.0-64.0; Al₂O₃: 10.0-25.0; B₂O₃: 0.0-10.0; La₂O₃: 0.1-10.0; WO₃: 5.0-14.0; ZrO₂: 0.1-7.0; MgO: 1.0-7.9; CaO: 0.1-6.0; ZnO: 0-6.0; SrO: 0-6.0, and by radically polymerizable compositions comprising at least one radically polymerizable monomer without acid groups, at least one radically polymerizable monomer comprising acid groups, at least one initiator for the radical polymerization and at least one radiopaque glass filler having the above composition. Compositions containing fillers are referred to as composites.

Unless otherwise stated, all percentages refer to the total mass of the glass, whereby the components are calculated as oxides, as is usual for glasses and glass ceramics.

The glass fillers according to the invention do not contain any alkali metal oxides or ions, i.e. in particular no Li₂O, Na₂O, K₂O, Rb₂O and Cs₂O, and preferably also no BaO.

Surprisingly, radiopaque glass fillers with the disclosed composition were found to be particularly acid stable, especially to the acidic adhesive monomers used in the manufacture of self-adhesive dental materials, without the need for acid treatment or other sophisticated surface treatment. They enable simplified production of storage-stable composite materials with self-adhesive properties.

The radiopaque glass fillers are preferably used without prior acid treatment, i.e. are in particular not washed with an organic or inorganic acid or a solution thereof. According to a particularly preferred embodiment of the invention, the radiopaque glass fillers are also used without any other prior surface treatment, in particular without a surface treatment according to the prior art discussed in the introduction.

However, the radiopaque glass fillers can be silanized to improve the bond between filler and matrix. Silanization is preferably carried out with a radically polymerizable silane, particularly preferably with a methacrylate-functionalized silane and most preferably with 3-methacryloyloxypropyltrimethoxy silane (MEMO). According to the invention, silanization is not a surface treatment in the above sense.

Preferred are radiopaque glass fillers with the following composition (wt. %): SiO₂: 55.0-63.0, particularly preferably 55.0-62.0; Al₂O₃: 12.0-24.0, particularly preferably 14.0-22.0; B₂O₃: 0.1-8.0, particularly preferably 0.1-5.9; La₂O₃: 0.1-8.0, particularly preferably 0.1-6.0; WO₃: 5.0-13.0, particularly preferably 5.0-12; ZrO₂: 0.1-6.0, particularly preferably 0.1-5.0; MgO: 2.0-7.5, particularly preferably 3.0-7.0; CaO: 0.1-4.0, particularly preferably 0.1-3.0; ZnO: 0-6.0, particularly preferably 0-4.0; SrO: 0-6.0, particularly preferably 0-5.0.

More preferred are radiopaque glass fillers having the following composition (wt. %): SiO₂: 56.0-60.0; Al₂O₃: 14.0-20.0; B₂O₃: 0.1-5.9; La₂O₃: 1.0-2.0; WO₃: 8.0-12; ZrO₂: 1.0-3.0; MgO: 5.0-7.0; CaO: 1.0-3.0; ZnO: 0; SrO: 0. Most preferred are glass fillers in which the sum of La₂O₃+WO₃ is in a range of 5.0 to 11.6 wt. % and the sum of Al₂O₃+La₂O₃+WO₃ are in a range of 23.6-31.6 wt. %.

The radiopaque glass fillers preferably have an average particle size of 0.2 to 20 μm and particularly preferably of 0.4 to 5 μm. Unless stated otherwise, all particle sizes herein are volume averaged particle sizes (D₅₀ values), i.e. 50% of the total volume of all particles is contained in particles having a diameter smaller than the stated value.

Particle size determination in the range of 0.1 μm to 1000 μm is preferably carried out by means of static light scattering (SLS), for example with a static laser scattering particle size analyzer LA-960 (Horiba, Japan) or with a Microtrac S100 particle size analyzer (Microtrac, USA). Here, a laser diode with a wavelength of 655 nm and an LED with a wavelength of 405 nm are used as light sources. The use of two light sources with different wavelengths enables the measurement of the entire particle size distribution of a sample in only one measurement run, whereby the measurement is carried out as a wet measurement. For this purpose, an aqueous dispersion of the filler is prepared and its scattered light is measured in a flow cell. The scattered light analysis for the calculation of particle size and particle size distribution is carried out according to the Mie theory according to DIN/ISO 13320. The measurement of the particle size in a range of 1 nm to 0.1 μm is preferably carried out by dynamic light scattering (DLS) of aqueous particle dispersions, preferably with a He-Ne laser with a wavelength of 633 nm, at a scattering angle of 90° and at 25° C., e.g. with a Malvern Zetasizer Nano ZS (Malvern Instruments, Malvern UK).

In the case of aggregated and agglomerated particles, the primary particle size can be determined from TEM images. Transmission electron microscopy (TEM) is preferably performed with a Philips CM30 TEM at an accelerating voltage of 300 kV. For sample preparation, drops of the particle dispersion are applied to a 50 Å thick copper grid (mesh size 300 mesh) coated with carbon and the solvent is subsequently evaporated. The particles are counted and the arithmetic mean is calculated.

The compositions according to the invention preferably contain from 10 wt. % to 80 wt. %, more preferably from 20 wt. % to 75 wt. % and most preferably from 30 wt. % to 70 wt. % of the radiopaque glass filler, based on the total mass of the composition.

In addition to the radiopaque glass fillers, the compositions according to the invention may contain further fillers.

Preferred further fillers are metal oxides, particularly preferably mixed oxides, which contain 60 to 80 wt. % SiO₂ and at least one of the metal oxides Yb₂O₃, ZnO, Ta₂O₅, Nb₂O₅ and/or La₂O₃, preferably Yb₂O₃ and/or ZnO, so that the total amount adds up to 100%. Mixed oxides are accessible e.g. by hydrolytic co-condensation of metal alkoxides. The metal oxides preferably have a mean particle size of 0.05 to 10 μm, particularly preferably of 0.1 to 5 μm.

Other preferred additional fillers are pyrogenic silica or precipitated silica with a primary particle size of 0.01-0.15 μm as well as quartz or glass ceramic powder with a particle size of 0.1 to 15 μm, preferably 0.2 to 5 μm, and ytterbium trifluoride. The ytterbium trifluoride preferably has a particle size of 80 to 900 nm and particularly preferably 100 to 300 nm.

In addition, so-called composite fillers are preferred as further fillers. These are also referred to as isofillers. These are splinter-shaped polymers which in turn contain a filler, preferably pyrogenic SiO₂, glass filler and/or ytterbium trifluoride. Preferred are polymers based on dimethacrylates. To produce the isofillers, the filler(s) are incorporated, for example, into a dimethacrylate resin matrix, the resulting composite paste is subsequently thermally polymerized and then ground.

A composite filler preferred according to the invention can be prepared, for example, by thermally curing a mixture of Bis-GMA (8.80 wt. %), UDMA (6.60 wt. %), 1,10-decanediol dimethacrylate (5.93 wt. %), dibenzoyl peroxide+2,6-di-tert.butyl-4-methylphenol (together 0.67 wt. %), glass filler (average particle size 0.4 μm; 53.0 wt. %) and YbF₃ (25.0 wt. %) and subsequent grinding of the cured material to the desired grain size. All percentages refer to the total mass of the composite filler.

So-called inertized fillers can also be used as additional fillers. These are glass fillers whose surface is coated with a diffusion barrier layer, e.g. on a sol-gel basis, or with a polymer layer, e.g. made of PVC. Preferred fillers are those described in EP 2 103 296 A1.

To improve the bond between filler and matrix, the further fillers are preferably surface modified with methacrylate functionalized silanes, such as 3-methacryloyloxypropyl trimethoxy silane.

The total amount of further fillers is preferably 0.1 to 25 wt. %, particularly preferably 0.2 to 20 wt. % and most preferably 0.3 to 15 wt. %, in each case based on the total mass of the composition. Particularly preferred according to the invention are compositions which contain 0.1 to 25 wt. %, preferably 1 to 20 wt. % and particularly preferably 2 to 15 wt. % of one or more of the said metal oxides, pyrogenic silica, precipitated silica or a mixture thereof, based on the total mass of the composition.

The compositions according to the invention contain at least one radically polymerizable monomer without acid groups and at least one radically polymerizable monomer with acid groups. Preferred compositions according to the invention comprise monomers with and monomers without acid groups in a weight ratio of from 1:5 to 1:36, more preferably from 1:6 to 1:25 and most preferably from 1:7 to 1:20.

Preferred according to the invention are compositions which contain at least one polyfunctional monomer and particularly preferably at least one monofunctional and at least one polyfunctional monomer. Polyfunctional monomers are understood to be monomers with two or more, preferably 2 to 4 and in particular 2 radically polymerizable groups. Monofunctional monomers accordingly have only one radically polymerizable group. Polyfunctional monomers have crosslinking properties and are therefore also referred to as crosslinking monomers. Preferred radically polymerizable groups are (meth)acrylate, (meth)acrylamide and vinyl groups.

According to the invention, a distinction is made between monomers containing acid groups and monomers which do not contain acid groups. Preferred monomers of these types are defined below.

### Monomers Without Acid Group

Preferred monomers without acid groups are (meth)acrylates, especially monofunctional and polyfunctional methacrylates and most preferably a mixture thereof. Preferred polyfunctional (meth)acrylates are difunctional methacrylates. Preferred monofunctional (meth)acrylates are benzyl, tetrahydrofurfuryl or isobornyl (meth)acrylate, p-cumyl-phenoxyethyleneglycol methacrylate (CMP-1E) and 2-([1,1′-biphenyl]-2-oxy)ethyl methacrylate (MA-836), tricyclodecanemethyl (meth)acrylate, 2-(2-biphenyloxy)ethyl (meth)acrylate. CMP-1E and MA-836 are particularly suitable.

According to one embodiment, the compositions according to the invention preferably comprise at least one functionalized monofunctional (meth)acrylate. Functionalized monomers are understood to be monomers which, in addition to at least one radically polymerizable group, carry at least one functional group, preferably a hydroxyl group. Preferred functionalized mono(meth)acrylates are 2-hydroxyethyl and hydroxyethyl-propyl (meth)acrylate and 2-acetoxyethyl methacrylate. Particularly preferred is 2-hydroxyethyl methacrylate. The monomers comprising acid groups mentioned below are not functionalized monomers within the meaning of the invention.

Preferred di- and polyfunctional (meth)acrylates are bisphenol-A-dimethacrylate, Bis-GMA (an addition product of methacrylic acid and bisphenol-A-diglycidyl ether), ethoxy- or propoxylated bisphenol-A-dimethacrylates, e.g. the bisphenol-A-dimethacrylate SR-348c (Sartomer) with 3 ethoxy groups or 2,2-bis[4-(2-methacryloyloxypropoxy)phenyl]propane, urethanes of 2-(hydroxymethyl)acrylic acid methyl ester and diisocyanates, such as 2,2,4-trimethylhexamethylene diisocyanate or isophorone diisocyanate, UDMA (an addition product of 2-hydroxyethyl methacrylate and 2,2,4-trimethyl hexamethylene-1,6-diisocyanate), tetramethylxylylene diurethane ethylene glycol di(meth)acrylate or tetramethylxylylene diurethane-2-methylethylene glycol di(meth)acrylate (V-380), di-, tri- or tetraethylene glycol dimethacrylate, trimethylolpropane trimethacrylate, pentaerythritol tetramethacrylate and glycerol di- and trimethacrylate, 1,4-butandiol dimethacrylate, 1,10-decanediol dimethacrylate (DsMA), bis(methacryloyloxymethyl)tricyclo-[5.2.1.0^{2,6}]decane (DCP), polyethylene glycol or polypropylene glycol dimethacrylates, such as polyethylene glycol 200 dimethacrylate or polyethylene glycol 400 dimethacrylate (PEG-200 or PEG-400 DMA) or 1,12-dodecanediol dimethacrylate. Bis-GMA, UDMA, V-380, triethylene glycol dimethacrylate (TEGDMA) and PEG-400-DMA (NK-ester 9G) are particularly preferred.

The monomer tetramethylxylylenediurethane ethylene glycol di(meth)acrylate or tetramethylxylylenediurethane-2-methylethylene glycol diurethane di(meth)acrylate (V-380) has the following formula:

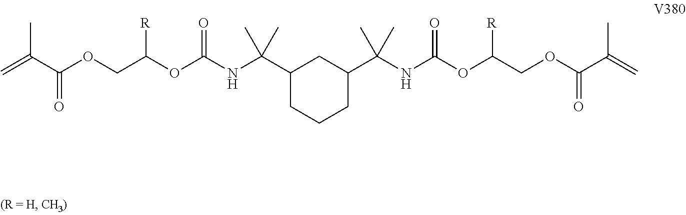

In the formula shown, the radicals R are each independently H or CH₃, where the radicals may have the same meaning or different meanings. Preferably, a mixture is used which contains molecules in which both radicals are H, molecules in which both radicals are CH₃, and molecules in which one radical is H and the other radical is CH₃, the ratio of H to CH₃ preferably being 7:3. Such a mixture is obtainable, for example, by reacting 1,3-bis(1-isocyanato-1-methylethyl)benzene with 2-hydroxypropyl methacrylate and 2-hydroxyethyl methacrylate.

Other preferred difunctional monomers are radically polymerizable pyrrolidones, such as 1,6-bis(3-vinyl-2-pyrrolidonyl)-hexane, or commercially available bisacrylamides such as methylene- or ethylene bisacrylamide, as well as bis(meth)acrylamides, such as N,N′-diethyl-1,3-bis(acrylamido)-propanes, 1,3-bis(methacrylamido)-propanes, 1,4-bis(acrylamido)-butanes or 1,4-bis(acryloyl)-piperazines, which can be synthesized by reacting the corresponding diamines with (meth)acrylic acid chloride. N,N′-diethyl-1,3-bis(acrylamido)propane (V-392) is particularly preferred. These monomers are characterized by a high hydrolysis stability.

### Monomers and Oligomers Comprising Acid Groups

The compositions according to the invention comprise at least one acidic, radically polymerizable monomer. Acidic monomers are understood to be monomers comprising at least one acid group, preferably a phosphoric ester, phosphonic acid or carboxyl group. Acidic monomers are also referred to herein as adhesive monomers. According to the invention, those compositions are particularly preferred which comprise at least one strongly acidic monomer. By strongly acidic monomers are meant monomers with a pKa value of 0.5 to 4.0, particularly preferably 1.0 to 3.5 and most preferably 1.5 to 2.5 at room temperature.

Suitable acid group-containing monomers are COOH group-containing polymerizable monomers, preferably with a pKₐ value in the range of 2.0 to 4.0. Preferred are 4-(meth)acryloyloxyethyl trimellitic anhydride, 10-methacryloyloxydecyl malonic acid, N-(2-hydroxy-3-methacryloyloxypropyl)-N-phenylglycine and 4-vinylbenzoic acid. Methacrylic acid (pKₐ=4.66) is excluded due to its low adhesion to the tooth structure.

Preferred monomers comprising acid groups are phosphoric ester and phosphonic acid monomers, preferably with a pKₐ value in the range of 0.5 to 3.5. Particularly preferred are 2-methacryloyloxyethylphenyl hydrogen phosphate, 10-methacryloyloxy-decyl dihydrogen phosphate (MDP), glycerol dimethacrylate dihydrogen phosphate or dipentaerythritol pentamethacryloyloxy phosphate, 4-vinylbenzylphosphonic acid, 2-[4-(dihydroxyphosphoryl)-2-oxa-butyl]acrylic acid or hydrolysis-stable esters such as 2-[4-(dihydroxyphosphoryl)-2-oxa-butyl]acrylic acid 2,4,6-trimethyl phenyl ester. MDP, 2-methacryloyloxyethylphenyl hydrogen phosphate and glycerol dimethacrylate dihydrogen phosphate are particularly preferred.

The compositions according to the invention may additionally contain one or more acidic radically polymerizable oligomers in addition to the monomer containing acid groups. However, compositions which do not contain acidic radically polymerizable oligomers are preferred. Oligomers are understood to be polymers with a degree of polymerization Pn of 2 to 100 (Pn=Mn/Mu; Mn: number average polymer molar mass, Mu: molar mass of the monomer unit). Acidic, radically polymerizable oligomers comprise at least one acid group, preferably a carboxyl group, and at least one radically polymerizable group, preferably at least one (meth)acrylate and particularly preferably at least one methacrylate group.

Oligomers containing acid groups preferred according to the invention are oligomeric carboxylic acids, such as polyacrylic acid, preferably with a number average molar mass Mn of less than 7200 g/mol, preferably less than 7000 g/mol and more preferably less than 6800 g/mol, wherein Mn is preferably in a range from 800 to 7200 g/mol, more preferably from 500 to 7000 g/mol and most preferably from 500 to 6800 g/mol. Particularly preferred are oligomeric carboxylic acids with (meth)acrylate groups. These can be obtained, for example, by reacting oligomeric polyacrylic acid with glycidyl methacrylate or 2-isocyanatoethyl methacrylate.

Unless otherwise stated, the molar mass of oligomers and polymers herein is the number average molar mass Mn determined by gel permeation chromatography (GPC).

Gel permeation chromatography (GPC) is a relative method in which molecules are separated on the basis of their size, more precisely on the basis of their hydrodynamic volume. The absolute molar mass is determined by calibration with known standards. Preferably, narrowly distributed polystyrene standards are used as calibration standards. These are commercially available. Styrene-divinylbenzene columns are used as separation material and tetrahydrofuran (THF) as eluent. Styrene-divinylbenzene columns are suitable for organic soluble synthetic polymers. The measurement is carried out with diluted solutions (0.05-0.2 wt. %) of the polymers to be investigated.

Alternatively, the number-average molar mass can be determined with the known methods of freezing point depression (cryoscopy), boiling point elevation (ebullioscopy) or from the depression of the vapor pressure (vapor pressure osmometry). These are absolute methods that do not require calibration standards. Concentration series of 4 to 6 diluted polymer solutions with concentrations of 0.005 to 0.10 mol/kg are examined and then the measured values are extrapolated to a concentration of 0 mol/kg.

The compositions according to the invention preferably contain water. It has been found that a water content of 1 to 7 wt. %, particularly preferably 1 to 5 wt. %, in each case based on the total mass of the composition, brings about an improvement in the bonding effect to dentin and enamel.

The compositions according to the invention further contain at least one initiator for initiating the radical polymerization, preferably a photoinitiator. Preferred photoinitiators are benzophenone, benzoin and derivatives thereof, α-diketones or derivatives thereof, such as 9,10-phenanthrenequinone, 1-phenyl propane-1,2-dione, diacetyl and 4,4-dichlorobenzil. Particularly preferred are camphorquinone (CC) and 2,2-dimethoxy-2-phenyl-acetophenone and most preferably α-diketones in combination with amines as reducing agents, such as ethyl 4-(dimethylamino)benzoate (EDMAB), N,N-dimethylaminoethyl methacrylate, N,N-dimethyl-sym.-xylidine or triethanolamine. Further preferred are Norrish type I photoinitiators, especially acyl or bisacyl phosphine oxides and most preferably monoacyltrialkylgermanium, diacyldialkylgermanium and tetraacylgermanium compounds, such as benzoyltrimethyl germane, dibenzoyl diethyl germane, bis(4-methoxybenzoyl)diethyl germane (Ivocerin®), tetrabenzoyl germane or tetrakis(o-methylbenzoyl) germane. Mixtures of the various photoinitiators can also be used, such as bis(4-methoxy benzoyl) diethyl germane or tetrakis(o-methylbenzoyl) germane in combination with camphorquinone and 4-dimethylaminobenzoic acid ethyl ester.

Further preferred are compositions comprising a redox initiator for initiating the radical polymerization, preferably a redox initiator based on an oxidizing agent and a reducing agent. Preferred oxidizing agents are peroxides and in particular hydroperoxides. A particularly preferred peroxide is benzoyl peroxide. Preferred hydroperoxides are the low-odor cumene hydroperoxide derivatives disclosed in EP 3 692 976 A1, the oligomeric CHP derivatives disclosed in EP 4 094 748 A1 (21315089.9) and in particular 4-(2-hydroperoxypropan-2-yl)phenylpropionate and cumene hydroperoxide (CHP).

Preferred reducing agents for combination with peroxides are tertiary amines, such as N,N-dimethyl-p-toluidine, N,N-dihydroxyethyl-p-toluidine, p-dimethylaminobenzoic acid ethyl esters or other aromatic dialkylamines, ascorbic acid, sulfinic acids, thiols and/or hydrogen silanes.

Preferred reducing agents for combination with hydroperoxides are thiourea derivatives, in particular the compounds listed in EP 1 754 465 A1 in paragraph [0009].

Particularly preferred are methyl-, ethyl-, allyl-, butyl-, hexyl-, octyl-, benzyl-, 1,1,3-trimethyl-, 1,1-diallyl-, 1,3-diallyl-, 1-(2-pyridyl-2-thio urea, acetyl, propanoyl, butanoyl, pentanoyl, hexanoyl, heptanoyl, octanoyl, nonanoyl, decanoyl, benzoylthiourea and mixtures thereof. Acetyl-, allyl-, pyridyl- and phenylthiourea as well as hexanoylthiourea and mixtures thereof as well as polymerizable thiourea derivatives, such as N-(2-methacryloyloxyethoxysuccinoyl)-thiourea and N-(4-vinyl benzoyl)-thiourea) are very particularly preferred. In addition, a combination of one or more of said thiourea derivatives with one or more imidazoles may advantageously be used. Preferred imidazoles are 2-mercapto-1-methylimidazole or 2-mercaptobenzimidazole.

In addition to at least one hydroperoxide and at least one thiourea derivative, the compositions according to the invention may also contain at least one transition metal compound for accelerating curing. Suitable transition metal compounds according to the invention are in particular compounds derived from transition metals having at least two stable oxidation states. Particularly preferred are compounds of the elements copper, iron, cobalt, nickel and manganese. These metals have the following stable oxidation states: Cu(I)/Cu(II), Fe(II)/Fe(III), Co(II)/Co(III), Ni(II)/Ni(III), Mn(II)/Mn(III). Compositions containing at least one copper compound are particularly suitable. The transition metal compounds are preferably used in catalytic amounts, particularly preferably in an amount of 10 to 200 ppm. These do not lead to discoloration of the dental materials. Due to their good monomer solubility, the transition metals are preferably used in the form of their acetyl acetonates, 2-ethyl hexanoates or THF adducts. Further preferred are their complexes with polydentate ligands such as 2-(2-aminoethylamino)ethanol, triethylenetetramine, dimethylglyoxime, 8-hydroxyquinoline, 2,2′-bipyridine or 1,10-phenanthroline. A particularly suitable initiator according to the invention is a mixture of cumene hydroperoxide (CHP) with at least one of the above-mentioned thiourea derivatives and copper(II) acetylacetonate. According to the invention, compositions which do not contain any vanadium compounds are preferred.

The compositions according to the invention preferably do not contain barbituric acid or barbituric acid derivatives such as 1,3,5-trimethylbarbituric acid, 1-benzyl-5-phenylbarbituric acid, 5-butylbarbituric acid or 1-cyclohexyl-5-ethylbarbituric acid. Compositions comprising barbiturates have unsatisfactory storage stability because barbiturates form polymerization-inducing radicals through oxidation with atmospheric oxygen. In addition, barbiturates have adverse physiological effects such as bradycardia, hypotension or blood disorders.

The compositions according to the invention may also contain further additives, in particular stabilizers, colorants, phase transfer catalysts, microbicidal agents, fluoride ion-donating additives, such as fluoride salts, in particular NaF or ammonium fluoride, or fluorosilanes, optical brighteners, plasticizers and/or UV absorbers.

According to the invention, such compositions are particularly preferred which comprise the following ingredients:
a) 10 to 80 wt. %, preferably 20 to 75 wt. % and particularly preferably 30 to 70 wt. % of at least one radiopaque glass filler according to the invention,
b) optionally 0.1 to 25 wt. %, preferably 1 to 20 wt. % and particularly preferably 2 to 15 wt. % of one or more further fillers,
c) 1 to 15 wt. %, preferably 2 to 12 wt. % and particularly preferably 3 to 10 wt. % of at least one monomer containing acid groups,
d) 5 to 40 wt. %, preferably 8 to 30 wt. % or 8 to 29 wt. % and particularly preferably 10 to 25 wt. % of at least one polyfunctional monomer without acid groups,
e) 0 to 10 wt. %, preferably 0 to 8 wt. % and particularly preferably 1 to 5 wt. % of one or more oligomeric carboxylic acids,
f) 1 to 20 wt. %, preferably 2 to 15 wt. % and particularly preferably 3 to 10 wt. % of one or more monofunctional monomers without acid groups,
g) 0.1 to 8 wt. %, preferably 0.5 to 6 wt. % and particularly preferably 1 to 5 wt. % of an initiator for the free radical polymerization,
h) 0 to 20 wt. %, preferably 0.2 to 10 wt. % and particularly preferably 1 to 7 wt. % of water and
i) 0.01 to 5 wt. %, preferably 0.1 to 3 wt. % and particularly preferably 0.1 to 2 wt. % of one or more additives, in each case based on the total mass of the composition.

The initiator can be a redox initiator, a photoinitiator or a combination thereof. The above quantities include all initiator components, i.e. the initiators themselves and, if applicable, reducing agents, transition metal compounds, etc. According to the invention, compositions containing at least one redox initiator or at least one redox initiator and at least one photo initiator are preferred.

Compositions containing a redox initiator are also called self-curing. They are preferably used in the form of two physically separated components, i.e. as a 2-component system (2K system). Oxidizing and reducing agents are incorporated into separate components of the composition. One component, the so-called catalyst paste, contains the oxidizing agent, preferably a peroxide or hydroperoxide, and the second component, the so-called base paste, contains the corresponding reducing agent and optionally a photoinitiator and optionally catalytic amounts of a transition metal compound. The polymerization is started by mixing the components. Compositions that contain both a redox and a photoinitiator are called dual-curing.

According to the invention, 2-component systems are preferred. They are preferably self-curing or dual-curing. The pastes are mixed together shortly before use, preferably with a double-push syringe. The catalyst paste preferably has the following composition:
a) 10 to 80 wt. %, preferably 20 to 75 wt. % and particularly preferably 30 to 70 wt. % of at least one acid-stable radiopaque glass filler,
b) optionally 0.1 to 25 wt. %, preferably 1 to 20 wt. % and particularly preferably 2 to 15 wt. % of one or more further fillers,
c) 2 to 30 wt. %, preferably 4 to 24 wt. % and particularly preferably 6 to 20 wt. % of at least one monomer containing acid groups,
d) 5 to 40 wt. %, preferably 8 to 30 wt. % or 8 to 29 wt. % and particularly preferably 10 to 25 wt. % of at least one polyfunctional monomer without acid groups,
e) 0 to 10 wt. %, preferably 0 to 8 wt. % and particularly preferably 1 to 5 wt. % of one or more oligomeric carboxylic acids,
f) 1 to 20 wt. %, preferably 2 to 15 wt. % and particularly preferably 3 to 10 wt. % of one or more monofunctional monomers without acid groups,
g) 0.01 to 16 wt. %, preferably 0.2 to 12 wt. % and particularly preferably 0.5 to 10 wt. % of at least one peroxide and/or hydroperoxide and optionally at least one photoinitiator,
h) 0 to 20 wt. %, preferably 0.2 to 10 wt. % and particularly preferably 1 to 7 wt. % water and
i) 0.001 to 5 wt. %, preferably 0.002 to 3 wt. % and particularly preferably 0.0051 to 2 wt. % of one or more additives, in each case based on the total mass of the catalyst paste.

The base paste preferably has the following composition:
a) 10 to 80 wt. %, preferably 20 to 75 wt. % and particularly preferably 30 to 70 wt. % of at least one radiopaque glass filler,
b) optionally 0.1 to 25 wt. %. preferably 1 to 20 wt. % and particularly preferably 2 to 15 wt. % of one or more further fillers,
d) 5 to 40 wt. %, preferably 8 to 30 wt. % or 8 to 29 wt. % and particularly preferably 10 to 25 wt. % of at least one polyfunctional monomer without acid groups,
f) 1 to 20 wt. %, preferably 2 to 15 wt. % and particularly preferably 3 to 10 wt. % of one or more monofunctional monomers without acid groups,
g) 0.01 to 16 wt. %, preferably 0.3 to 12 wt. % and particularly preferably 1 to 10 wt. % of at least one appropriate reducing agent and optionally at least one photoinitiator,
h) 0 to 20 wt. %, preferably 0.2 to 10 wt. % and particularly preferably 1 to 7 wt. % water and
i) 0.001 to 5 wt. %, preferably 0.002 to 3 wt. % and particularly preferably 0.0051 to 2 wt. % of one or more additives, in each case based on the total mass of the base paste.

For application, the catalyst and base paste are preferably mixed together in approximately equal proportions. They are therefore particularly suitable for use with a double-barrel syringe. Double-barrel syringes have two separate cylindrical chambers for holding the base and catalyst paste. The components are pressed out of the chambers simultaneously with two connected pistons and are preferably pressed through a mixing cannula and mixed together therein. To press out the pastes, the syringe can be inserted into a so-called hand dispenser, which facilitates the handling of the syringes.

It was surprisingly found that the radiopaque glass fillers according to the invention exhibit high acid stability even without prior surface treatment, especially with respect to the acidic adhesive monomers and oligomers used for the production of self-adhesive dental materials. They can be mixed directly with the other components of the materials and produce storage-stable composite materials with self-adhesive properties. During storage, the content of acid group-containing, radically polymerizable monomers decreases only very slowly, and the compositions show high adhesion to the tooth structure and especially to dentin even after prolonged storage.

The compositions according to the invention are also characterized by improved transparency. This is preferably greater than 10%. They are particularly suitable as dental materials for intraoral use by the dentist for the restoration of damaged teeth (therapeutic use), in particular as dental cements, coating or veneering materials, filling composites and especially as luting cements. The transparency is measured in transmission by means of a spectrophotometer, for example a Konika-Minolta CM-5 spectrophotometer, on 1 mm thick test specimens polished to a high gloss.

To treat damaged teeth, they are preferably prepared by the dentist in a first step. Subsequently, at least one composition according to the invention is applied onto or into the prepared tooth. Thereafter, the composition can be cured directly, preferably by irradiation with light of a suitable wavelength, for example when restoring cavities. Alternatively, a dental restoration, for example an inlay, onlay, veneer, crown, bridge, framework or dental ceramic, is placed in or applied to the prepared tooth. The subsequent curing of the composition is preferably achieved by light and/or self-curing. Thereby, the dental restoration is attached to the tooth.

The compositions according to the invention can also be used as extraoral materials (non-therapeutic use), for example in the manufacture or repair of dental restorations. They are also suitable as materials for the fabrication and repair of inlays, onlays, crowns or bridges.

For the production of dental restorations such as inlays, onlays, crowns or bridges, at least one composition according to the invention is formed into the desired dental restoration in a manner known per se and then hardened. The curing can be done by light, self-curing or preferably thermally.

In the repair of dental restorations, the compositions according to the invention are applied to the restoration to be repaired, for example to repair gaps or to reduce fragments, and then cured.

The invention is explained in more detail below with the aid of figures and examples.

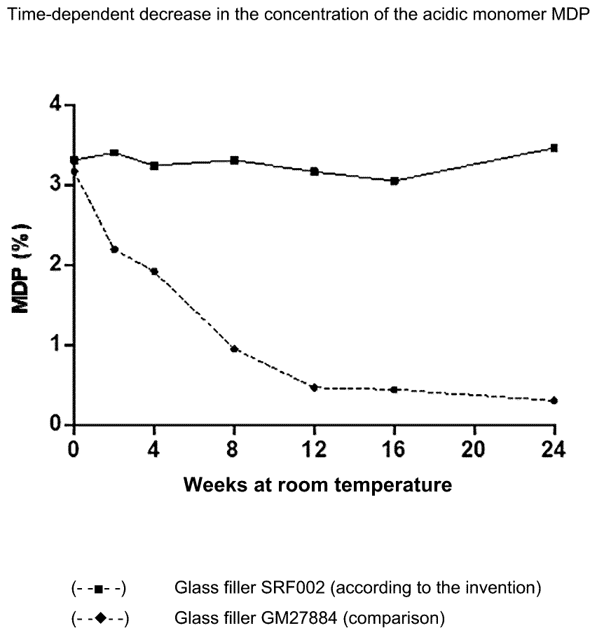

FIG. 1 shows the decrease in the concentration of the acid monomer MDP as a function of the storage time in composite pastes with an acid stable radiopaque glass filler ( [symbol] ) according to the invention and a conventional radiopaque glass filler ( [symbol] ).

### EXAMPLES

### Example 1

### Preparation of Acid-Stable Fillers

To prepare glass fillers, a homogeneous mixture of approx. 120 g of the oxides listed in Table 1 was prepared in a laboratory mixer (SpeedMixer®, Hauschild GmbH & Co. KG, Hamm). Afterwards, the raw material mixture was melted at 1650° C. in a Pt/Rh10 crucible and homogenized for 1.5 h. The molten glass was then quenched in water to produce a glass frit. The glass frit was comminuted by means of a counter-jet mill to a medium particle size of D₅₀ of approx. 5 μm. The composition of the produced glasses SFR001 to SFR005 is shown in Table 1.

TABLE 1
Composition (wt. %) of the glasses SRF001-005

| | Glass Filler | | | | |
|---|---|---|---|---|---|
| Oxide | 001 | 002 | 003 | 004 | 005 |
| SiO₂ | 58.0 | 58.0 | 58.0 | 58.0 | 58.0 |
| Al₂O₃ | 14.0 | 16.0 | 12.0 | 18.0 | 20.0 |
| B₂O₃ | 6.0 | 4.0 | 8.0 | 2.0 | 0.0 |
| La₂O₃ | 1.6 | 1.6 | 1.6 | 1.6 | 1.6 |
| WO₃ | 10.0 | 10.0 | 10.0 | 10.0 | 10.0 |
| ZrO₂ | 2.0 | 2.0 | 2.0 | 2.0 | 2.0 |
| MgO | 6.4 | 6.4 | 6.4 | 6.4 | 6.4 |
| CaO | 2.0 | 2.0 | 2.0 | 2.0 | 2.0 |
| Σ | 100.0 | 100.0 | 100.0 | 100.0 | 100.0 |

For silanization of the glass fillers, 6 g of 3-methacryloyloxypropyl trimethoxysilane (Silane A-174, Sigma Aldrich) was added to each of the fillers (90 g) and then mixed for 15 min (Turbola mixer, Willy A. Bachofen AG). Then 2.5 g deionized water was added and mixed again for 15 min. Afterwards, the fillers were sieved through a 90-μm plastic sieve, left to rest for 24 h and then dried in a drying cabinet at 50° C. for 3 days until no more free silane was detectable (gas chromatography).

### Example 2

### Investigation of the Storage Stability of Composite Pastes Based on the Filler SRF002 (Silanized) According to the Invention and a Common Radiopaque Glass Filler GM 27884

With the filler SRF002 (silanized) according to the invention from Example 1 and a radiopaque, dental glass filler (GM 27884, Schott company, mean particle size 1 μm, specific surface area (BET DIN ISO 9277) 3.9 m²/g, composition (wt. %): Al₂O₃: 10, B₂O₃: 10, BaO: 25 and SiO₂: 55) composite pastes were prepared. The compositions (wt. %) of the composite pastes are given in Table 2.

TABLE 2
Composition of the composite pastes (wt. %)

Composite

| Component | EC52-0981 | EC31-0221*) |
|---|---|---|
| MDP¹⁾ | 3.50 | 3.29 |
| TEGDMA²⁾ | 11.37 | 18.68 |
| NK ester 9G³⁾ | 2.53 | 2.37 |
| V-392⁴⁾ | 15.57 | 14.63 |
| BHT⁵⁾ | 0.03 | 0.03 |
| Glass filler SRF002 sil. | 65.00 | — |
| (Ex. 1) | | |
| Glass filler GM27884 sil. | — | 65.00 |
| Aerosil 200⁶⁾ | 2.00 | — |
| HDK 2000⁷⁾ | — | 4.00 |

*)Comparative example
¹⁾MDP (10-methacryloyloxydecyl dihydrogen phosphate, Orgentis)
²⁾TEGDMA (triethylene glycol dimethacrylate)
³⁾NK-Ester 9G (polyethylene glycol-400-dimethacrylate, Kowa Europa GmbH)
⁴⁾V-392 (N,N′-diethyl-1,3-bis(acrylamido)propane, Ivoclar Vivadent AG)
⁵⁾BHT (2,6-di-tert-butyl-p-cresol)
⁶⁾Aerosil 200, pyrogenic SiO₂, spec. surface area 200 m²/g (Evonik)
⁷⁾HDK 2000, pyrogenic SiO₂, spec. surface area 200 m²/g (Wacker)

The pastes were stored at room temperature and over a period of several weeks the change in MDP content over time was determined using ³¹P-NMR spectroscopy. For the ³¹P-NMR measurement, 1.5 g of the composite sample to be analyzed was weighed into a centrifuge tube and 1.5 ml of a solution of 0.1 g triphenylphosphine in 10 ml deuterated acetone (acetone-d₆) was added as an internal standard. The resulting suspension was shaken for at least 5 min using a platform shaker (Vibramax, Heidolph Instruments GmbH & Co. KG, Schwabach) and then centrifuged at 3500 rpm for 15 min. The supernatant was transferred into an NMR sample tube using a disposable pipette. The ³¹P-NMR measurement was performed with a 400 MHz nuclear magnetic resonance spectrometer (Avance DPX 400, Bruker Spectrospin). The MDP concentration was calculated from the ratio of the peak areas in relation to the inner standard. FIG. 1 shows the change in MDP content over time. The results in FIG. 1 demonstrate a significantly improved storage stability of the paste based on the filler SRF002 ( [symbol] ) according to the invention compared to the dental glass filler GM 27884 ( [symbol] ).

1. An X-ray opaque glass filler, comprising in wt. %: SiO2: 54.0-64.0; Al2O3: 10.0-25.0; B2O3: 0.0-10.0; La2O3: 0.1-10.0; WO3: 5.0-14.0; ZrO2: 0.1-7.0; MgO: 1.0-7.9; CaO: 0.1-6.0; ZnO: 0-6.0; SrO: 0-6.0, with all percentages referring to the total mass of the glass and all components calculated as oxides.

2. The glass filler of claim 1, comprising in wt. %: SiO₂: 55.0-63.0; Al₂O₃: 12.0-24.0; B₂O₃: 0.1-8.0; La₂O₃: 0.1-8.0; WO₃: 5.0-13.0; ZrO₂: 0.1-6.0; MgO: 2.0-7.5; CaO: 0.1-4.0; ZnO: 0-6.0; SrO: 0-6.0, with all percentages referring to the total mass of the glass and all components being calculated as oxides.

3. The glass filler according to claim 1, which does not contain alkali metal oxides.

4. The glass filler according to claim 1, which is not surface-treated.

5. A radically polymerizable composition comprising at least one radically polymerizable monomer without an acid group, at least one radically polymerizable monomer containing an acid group, a glass filler and at least one initiator for the radical polymerization, characterized in that the composition comprises at least one glass filler according to claim 1.

6. The composition according to claim 5, which comprises
a) 10 to 80 wt. % of the radiopaque glass filler,
b) optionally 0.1 to 25 wt. % of one or more further fillers,
c) 1 to 15 wt. % of at least one monomer containing acid groups,
d) 5 to 40 wt. % of at least one polyfunctional monomer without acid groups,
e) 0 to 10 wt. % of one or more oligomeric carboxylic acids,
f) 1 to 20 wt. % of one or more monofunctional monomers without acid groups,
g) 0.1 to 8 wt. % of an initiator for the radical polymerization,
h) 0 to 20 wt. % water and
i) 0.01 to 5 wt. % of one or more additives, in each case based on the total mass of the composition.

7. The composition according to claim 5, comprising as the radically polymerizable monomer at least one polyfunctional monomer selected from bisphenol A-dimethacrylate, bis-GMA (an addition product of methacrylic acid and bisphenol A diglycidyl ether), ethoxy- or propoxylated bis phenol-A-dimethacrylates, bisphenol-A-dimethacrylate with 3 ethoxy groups, 2,2-bis[4-(2-methacryloyloxypropoxy)phenyl]propane, UDMA (addition product of 2-hydroxyethyl methacrylate and 2,2,4-trimethylhexamethylene-1,6-diisocyanate), tetramethylxylylene diurethane ethylene glycol di(meth)acrylate, tetramethylxylylene diurethane-2-methylethylene glycol diurethane di(meth)acrylate (V-380), di-, tri- or tetraethylene glycol dimethacrylate, trimethylolpropane trimethacrylate, pentaerythritol tetramethacrylate, glycerol di- and glycerol trimethacrylate, 1,4-butanediol dimethacrylate, 1,10-decanediol dimethacrylate (D₃MA), bis(methacryloyloxy methyl) tricyclo-[5.2.1.0^{2,6}]decane (DCP), polyethylene glycol or polypropylene glycol dimethacrylates, polyethylene glycol 200 dimethacrylate (PEG-200-DMA), polyethylene glycol 400 dimethacrylate (PEG-400-DMA), 1,12-dodecanediol dimethacrylate, urethanes of 2-(hydroxymethyl)acrylic acid and diisocyanates, 2,2,4-trimethylhexamethylene diisocyanate, isophorone diisocyanate, pyrrolidones, 1,6-bis(3-vinyl-2-pyrrolidonyl)hexane, bisacrylamides, methylene bisacrylamide, ethylene bisacrylamide, bis(meth)acrylamides, N,N′-diethyl-1,3-bis(acrylamido)propane, 1,3-bis(methacrylamido)propane, 1,4-bis(acrylamido)butane, 1,4-bis(acryloyl)piperazine and mixtures thereof.

8. The composition according to claim 5, which comprises as the acid group-containing monomer at least one monomer with a pKa value of 0.5 to 4.0 at room temperature.

9. The composition according to claim 5, comprising as the acid group containing monomer at least one monomer selected from monomers containing a phosphoric ester group or phosphonic acid group, 2-methacryloyloxyethylphenylhydrogen phosphate, 10-methacryloyloxydecyldihydro gene phosphate (MDP), glycerol dimethacrylate dihydrogen phosphate, dipentaerythritpentamethacryloyloxy phosphate, 4-vinyl benzyl phosphonic acid, 2-[4-(dihydroxyphosphoryl)-2-oxa-butyl]acrylic acid, 2-[4-(dihydroxyphosphoryl)-2-oxa-butyl]acrylic acid 2,4,6-trimethylphenyl ester, 4-(meth)acryloyloxyethyltrimellitic anhydride, 10-methacryloyloxydecyl malonic acid, N-(2-hydroxy-3-methacryloyloxypropyl)-N-phenylglycine, 4-vinylbenzoic acid or a mixture thereof.

10. The composition according to claim 6, which comprises as the one or more oligomeric carboxylic acids (e) polyacrylic acid having a number-average molar mass of less than 7200 g/mol.

11. The composition according to claim 5, comprising as the radically polymerizable monomer at least one monofunctional monomer selected from benzyl (meth)acrylate, tetrahydrofurfuryl (meth)acrylate, isobornyl (meth)acrylate, p-cumylphenoxyethylene glycol methacrylate (CMP-1E), 2-([1,1′-biphenyl]-2-oxy)ethyl methacrylate (MA-836), tricyclodecanemethyl (meth)acrylate, 2-(2-biphenyloxy)ethyl (meth)acrylate, 2-hydroxyethyl (methacrylate), hydroxyethylpropyl (methacrylate), 2-acetoxyethyl methacrylate or a mixture thereof.

12. The composition according to claim 5, comprising a catalyst paste and a base paste, wherein the catalyst paste comprises
a) 10 to 80 wt. % of the radiopaque glass filler,
b) optionally 0.1 to 25 wt. % of one or more further fillers,
c) 2 to 30 wt. % of at least one monomer containing acid groups,
d) 5 to 40 wt. % of at least one polyfunctional monomer without acid groups,
e) 0 to 10 wt. % of one or more oligomeric carboxylic acids,
f) 1 to 20 wt. % of one or more monofunctional monomers,
g) 0.01 to 16 wt. % of at least one peroxide and/or hydroperoxide and optionally at least one photoinitiator,
h) 0 to 20 wt. % water and
i) 0.001 to 5 wt. % of one or more additives, in each case based on the total mass of the catalyst paste,
and where the base paste comprises
a) 10 to 80 wt. % of the radiopaque glass filler,
b) optionally 0.1 to 25 wt. % of one or more further fillers,
d) 5 to 40 wt. % of at least one polyfunctional monomer without acid groups,
f) 1 to 20 wt. % of one or more monofunctional monomers without acid groups,
g) 0.01 to 16 wt. % of at least one appropriate reducing agent and optionally at least one photoinitiator,
h) 0 to 20 wt. % water and
i) 0.001 to 5 wt. % of one or more additives, in each case based on the total mass of the base paste.

13. The composition according to claim 5 for therapeutic use as a dental material.

14. The composition according to claim 13, wherein the dental material comprises a dental cement, coating material, veneering material, filling composite or luting cement.

15. A process of manufacturing or repairing dental restorations comprising using the composition according to claim 5 to manufacture or repair dental restorations.

16. The process according to claim 15, wherein the dental restorations comprise inlays, onlays, crowns or bridges.

17. A process of manufacturing a dental material comprising mixing the radiopaque glass according to claim 1 with other components to produce the dental material.
